(19) United States
(12) Patent Application Publication
(10) Pub. No.: US 2007/0010508 A1
(43) Pub. Date: Jan. 11, 2007

(54) Use of 10-hydroxy-10,11-dihydrocarbamazepine derivatives for the treatment of affective disorders

(76) Inventors: Ferenc Martenyi, Budapest (HU); Markus Schmutz, Schoenenbuch (CH); Stefanie Zechner, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/550,382

(22) PCT Filed: Apr. 1, 2004
(86) PCT No.: PCT/EP04/03590

§ 371 Date: Aug. 11, 2006

Related U.S. Application Data
(60) Provisional application No. 60/459,864, filed on Apr. 2, 2003.

(51) Int. Cl. A61K 31/55

(52) U.S. Cl. 514/217

(57) ABSTRACT

The present invention relates to a method for the treatment, including the maintenance treatment, of affective disorders, which comprises administering, especially every 20 to 28 hours, to said subject a therapeutically effective amount, especially an amount between about 500 and about 3000 mg, of a compound of formula (1) wherein R₁ represents hydrogen or C₁-C₃alkyl carbonyl; to the use of a compound of (1) or a pharmaceutically acceptable salt thereof for the manufacture of a pharmaceutical composition for the treatment of affective disorders; to a combination comprising: (a) a compound of formula (I); and (b) at least one compound selected from the group consisting of lithium, divalproex, conventional antipsychotics, atypical antipsychotics, lamotrigine and antidepressants; and to the use of such combination for the treatment of affective disorders.

The present invention relates to new pharmaceutical uses of 10-hydroxy-10,11-dihydrocarbamazepine derivatives and combinations comprising said compounds.

In particular, the invention relates to a method for the treatment of affective disorders in a subject in need of such treatment, which comprises administering to said subject a therapeutically effective amount of a compound of formula I 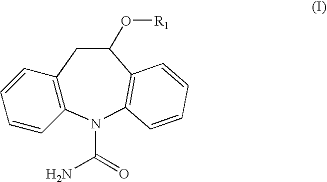
wherein R, represents hydrogen or C₁-C₃alkyl carbonyl, alone or in combination with further therapeutically active compounds as specified herein.

The preparation of the compound of formula I wherein R₁ is hydrogen and its pharmaceutically acceptable salts is described, e.g., in U.S. Pat. No. 3,637,661. Such compound, 10-hydroxy-10,11-dihydro-carbamazepine, the main metabolite of the antiepileptic oxcarbazepine (Trileptal®) is well known from the literature [see for example Schuetz H. et al., Xenobiotica (GB), 16(8), 769-778 (1986)]. The compound is indicated to be suitable for the treatment of psychosomatic disturbances, epilepsy, trigeminal neuralgia and cerebral spasticity.

The preparation of the compound of formula I wherein R₁ is C₁-C₃alkyl carbonyl and its pharmaceutically acceptable salts is described, e.g., in U.S. Pat. No. 5,753,646. The compounds are described to be efficacious against epilepsy.

Bipolar disorder is a chronic and severe disorder that affects approximately 1 % of the adult population. Bipolar I disorder is characterized by manic or ‘mixed’ mood states alternating with discrete periods of major depression or euthymia. Manic episodes may be manifested by elevated, expansive, or irritable mood, often accompanied by hyperactivity, insomnia, agitation, pressured speech, and disorganized thinking. Thought disorder may often be of psychotic proportions. In ‘mixed’ mood states both manic and depressive symptoms co-exist, with patients moving rapidly between sadness, irritability, and euphoria. The inflation of self-esteem and reduction in insight that often accompany these episodes is commonly manifested as poor judgment, leading to regrettable actions in interpersonal, occupational, or sexual arenas. As such, the social and economic impact of the disease is far-reaching, with a patient's livelihood and relationships as casualties; suicide attempts are common, with fatal outcomes in 10-15% of attempts.

The currently available treatments have substantial limitations. Primarily, 30-40% of manic patients do not show a substantial reduction in acute manic symptoms, let alone remission of symptoms, even after several weeks of treatment. In addition, many patients are unable to tolerate the adverse events deriving from the currenty available antimanic agents, these adverse events include nausea, extra-pyramidal symptoms (EPS), weight gain, sedation, cognitive dulling, fatigue and sexual dysfunction. Indeed, such side effects may be partially responsible for the high rate of non-compliance observed with available treatment. Finally, routine clinical monitoring for potentially severe or fatal side effects must accompany the use of several agents (e.g., renal and thyroid toxicity with lithium, hepatotoxicity and pancreafitis with valproate, and acute extrapyramidal symptoms and tardive dyskinesia with typical antipsychotics). Thus, new therapeutic agents are needed which offer improved safety and tolerability profiles along with efficacy in both the manic and depressive phases of bipolar disorder.

In accordance with the present invention, it has now surprisingly been found that a compound of formula I and the pharmaceutically acceptable salts thereof is useful for the treatment of affective disorders, in particular bipolar disorder.

Hence, the present invention provides a method for the treatment of affective disorders in a subject in need of such treatment, which comprises administering to said subject a therapeutically effective amount of a compound of formula I 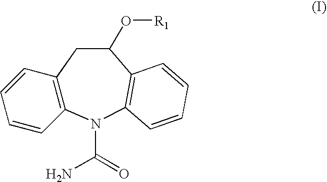
wherein R₁ represents hydrogen or C₁-C₃alkyl carbonyl.

Furthermore, the present invention relates to a method for the treatment of affective disorders, for example severe acute mania or bipolar disorders, in a subject in need of such treatment, which comprises administering to said subject every 20 to 28 hours an amount between about 500 and about 3000 mg, preferably between 750 and 2500 mg, or, in the case of severe acute mania, between 1500 and 2500 mg of a compound of formula I wherein R₁ represents hydrogen or C₁-C₃alkyl carbonyl.

Additionally, the present invention pertains to a method for the maintenance treatment of affective disorders in a subject in need of such treatment, which comprises administering to said subject a therapeutically effective amount of a compound of formula I wherein R₁ represents hydrogen or C₁-C₃alkyl carbonyl.

In one embodiment, the present invention pertains to a method for the maintenance treatment of affective disorders in a subject in need of such treatment, which comprises administering to said subject every 20 to 28 hours an amount between about 600 and about 2500 mg, preferably between 750 and 1250 mg, of a compound of formula I wherein R₁ represents hydrogen or C₁-C₃alkyl carbonyl.

In one preferred embodiment of the present invention, R₁ represents hydrogen.

In another preferred embodiment of the present invention, R₁ represents acetyl.

In particular, the present invention provides new therapies that offer advances in terms of safety and tolerability compared to existing treatments resulting, e.g., in increased patient acceptance and compliance.

The compounds of formula I constitute chiral compounds. For the purposes of the present invention, the chiral compounds disclosed herein can be employed in the form of racemates, in mixtures comprising one enantiomer in excess (e.g., more S-10-hydroxy-10,11-dihydro-carbamazepine than R-10-hydroxy-10,11-dihydro-carbamazepine) or in enantiomerically pure form (e.g. pure S-10-hydroxy-10,11-dihydro-carbamazepine or pure S-10-acetoxy-10,11-dihydro-carbamazepine).

The pure enantiomers of a compound of formula I can be obtained starting from the corresponding racemates by procedures known as such. The racemates may be separated into the enantiomers through the formation of diastereomeric salts, for example by salt formation with an enantiomer-pure chiral acid, or by means of chromatography, for example by HPLC, using chromatographic substrates with chiral ligands.

In one embodiment of the invention, the pure enantiomers of the compound of formula I wherein R₁ represents hydrogen are prepared according to the procedures described in the Examples.

The pure enantiomers of the compound of formula I wherein R₁ represents C₁-C₃alkyl carbonyl can be prepared, e.g., according to the procedures described in U.S. Pat. No. 5,753,646 or WO02/09257.

The term “enantiomerically pure form” as used herein means that a chiral compound is almost free of its enantiomer, i.e., a sample of the chiral compound comprises less than about 5, preferably less than about 2, more preferably less than about 0.5, weight percent of its enantiomer.

Hence, in one embodiment the present invention relates to the use of a compound of formula I, wherein R₁ represents hydrogen or C₁-C₃alkyl carbonyl, especially acetyl, wherein the compound is employed in enantiomerically enriched or pure form.

In a preferred embodiment of the invention, the compound of formula I, wherein R₁ represents hydrogen, is employed in the form of a racemate, i.e. a 1:1 mixture of both enantiomers.

The term “affective disorders” as used herein includes, but is not limited to uni- and bipolar depression, bipolar disorder, pre-menstrual dysphoric disorder, post-partum depression, post-menopausal depression, neurodegeneration-related depressive symptoms and depression occurring following cessation of psychostimulant intake, psychotic states, e.g. mania, schizophrenia, and excessive mood swings where behavioural stabilization is desired. One preferred embodiment of the present invention relates to the treatment of manic episodes of bipolar I disorder. Another preferred embodiment of the present invention relates to the treatment of acute mania in patients with a history of rapid cycling, with psychotic features, euphoric mania or dysphoric mania. Another aspect of the present invention is the treatment of manic symptoms.

Administration “every 20 to 28 hours” means preferably administration every 22 to 26 hours, more preferably about every 24 hours.

The pharmacological activity of a compound of formula I may, for example, be demonstrated in clinical studies. Such clinical studies are preferably randomized, double-blind, clinical studies in 300 to 500 patients, e.g. 400, 430 or 450 patients, with affective disorders comprising administering a compound of formula I wherein R₁ is hydrogen in a total daily dose between 750 and 2500 mg. In such study, the compound of formula I wherein R₁ is hydrogen can be applied, e.g., in the form of oral tablets having dose strengths 125 mg, 250 mg and 500 mg. The beneficial effects on affective disorders can be determined directly through the results of these studies or by changes in the study design which are known as such to a person skilled in the art. Efficacy can be measured, e.g. by a change in total score of the Young Mania Rating Scale (Y-MRS) from baseline to week 6. Such a scenario is in particular suitable to demonstrate efficacy of the treatment using a compound of formula I, wherein R₁ is hydrogen, in a wide range of bipolar patients (rapid cycling/non rapid cycling, with/without psychotic features, mixed/euphoric mania).

The activity of the compound of formula I in the treatment of affective disorders treatment is also evidenced, for example, in tests suitable for detecting drugs having potential behavioural desinhibitory and/or sociotropic effects which are thought to be relevant for recovery from social withdrawal, a cardinal feature of depression and related psychiatric conditions. For instance, drug effects on social withdrawal of intruder mice can be evaluated by using the basic method as described in Triangle, 1982, 21 :95105 and J. Clin. Psychiatry, 1994, 55:9 (suppl. B) 4-7.

Furthermore, the activity of the compound of formula I in the treatment of affective disorders treatment can be evidenced in the Vogel conflict test. The Vogel conflict test is the standard test to detect psychiatric, mainly anxiolytic and antidepressant drug action since various classes of anxiolytic and antidepressant drugs are effective in this test and since there is a high co-morbidity between anxiety states and other psychiatric, e.g., depression disorders. The surprising high efficacy of a compound of formula I in this test is therefore indicative of drug activity in depression or other affective disorders as defined above.

The compounds may be administered in any usual manner, e.g. orally, for example in the form of tablets or capsules, or parenterally, for example in the form of injection solutions or suspensions.

The present invention also provides pharmaceutical compositions comprising the compounds in association with at last one pharmaceutical carrier or diluent for use in the treatment of affective disorders. Such compositions may be manufactured in conventional manner.

The invention further provides the use of a compound of formula I for the manufacture of a pharmaceutical composition for the treatment of affective disorders.

For the treatment of conditions associated with affective disorders, appropriate dosage will of course vary depending upon, for example, the host, the mode of administration and the nature and severity of the condition being treated. In larger mammals, for example humans, an indicated daily dosage is in the ranges as provided above, conveniently administered, for example, in divided doses up to four times a day.

Unit dosage forms may contain for example from about 2.5 mg to about 1000 mg of the compound, preferably about 300 or 600 mg.

For the treatment of affective disorders a compound of formula I can be administered alone or in combination with at least one compound selected from the group consisting of lithium, valproic acid sodium salt, conventional antipsychotics, atypical antipsychotics, lamotrigine and antidepressants, in which the active ingredients are present in each case in free form or in the form of a pharmaceutically acceptable salt, and optionally at least one pharmaceutically acceptable carrier.

The term “lithium” as used herein includes, but is not limited to lithium acetate, lithium carbonate, lithium chloride, lithium citrate and lithium sulfate.

The term “conventional antipsychotics” as used herein includes, but is not limited to haloperidol and fluphenazine.

The term “atypical antipsychotics” as used herein includes, but is not limited to olanzapine, quetiapine, risperidone and aripiprazol.

The term “antidepressants” as used herein includes, but is not limited to selective serotonin reuptake inhibitors (SSRI's). An SSRI's suitable for the present invention is especially selected from fluoxetine, fluvoxamine, sertraline, paroxetne and escitalopram.

Lithium acetate can be administered, e.g., in the form as marketed, e.g. under the trademark Quilonorm™. Lithium carbonate can be administered, e.g., in the form as marketed, e.g. under the trademark Eskalith™. Lithium citrate can be administered, e.g., in the form as marketed, e.g. under the trademark Litarex™. Lithium sulfate can be administered, e.g., in the form as marketed, e.g. under the trademark Lithium-Duriles™. Valproic acid sodium salt can be administered, e.g., in the form as marketed, e.g. under the trademark Divalproex Sodium™. Haloperidol can be administered, e.g., in the form as marketed, e.g. under the trademark Haloperidol STADA™. Fluphenazine can be administered, e.g., in the form of its dihydrochloride as marketed, e.g. under the trademark Prolixin™. Lamotrigine can be administered, e.g., in the form as marketed, e.g. under the trademark Lamictal™. Olanzapine can be administered, e.g., in the form as marketed, e.g. under the trademark Zyprexa™. Risperidone can be administered, e.g., in the form as marketed, e.g. under the trademark Risperdal™. Aripiprazol can be administered, e.g., in the form as marketed, e.g. under the trademark Abilify™ or in any form as desribed in U.S. Pat. No. 5,006,528, which is included herein by reference. Quetiapine can be administered, e.g., in the form as marketed, e.g. under the trademark Seroquel™. Fluoxetine can be administered, e.g., in the form of its hydrochloride as marketed, e.g. under the trademark Prozac™. Fluvoxamine can be administered as free base or in the form of the maleate, e.g., in the form as marketed, e.g. under the trademark Fevarin™, Luvox™, Faverin™ or Depromel™. Paroxetine can be administered, e.g., in the form as marketed, e.g. under the trademark Paxil™. Escitalopram can be administered as free base or in the form of the oxalate or propanoate, e.g., in the form as marketed, e.g. under the trademark Lexapro™.

The structure of the active agents identified by code nos., generic or trade names may be taken from the actual edition of the standard compendium “The Merck Indek” or from databases, e.g. Patents International (e.g. IMS World Publications). The corresponding content thereof is hereby incorporated by reference. For example, sertraline can be prepared as disclosed in U.S. Pat. No. 4,536,518 to Pfizer.

The pharmacological activity of a combination as disclosed herein may, for example, be demonstrated in clinical studies. Such clinical studies are preferably randomized, double-blind, clinical studies in patients with affective disorders. Such studies demonstrate, in particular, the synergism of the active ingredients of the combination as disclosed herein. The beneficial effects on affective disorders can be determined directly through the results of these studies or by changes in the study design which are known as such to a person skilled in the art. The studies are, in particular, suitable to compare the effects of a monotherapy using the active ingredients and those of a combination as disclosed herein. Efficacy can be measured, e.g. by a change in total score of the Young Mania Rating Scale (Y-MRS) from baseline to week 6.

One suitable clinical study design would, e.g., be a combination study, wherein a compound of formula I wherein R₁ is hydrogen, is employed in combination with lithium or olanzapine. Such a scenario is in particular suitable to demonstrate superior efficacy of the treatment using a compound of formula I wherein R₁ is hydrogen plus lithium or olanzapine compared to lithium or olanzapine monotherapy in a wide range of bipolar patients (rapid cycling/non rapid cycling, with/without psychotic features, mixed/euphoric mania), as well as safety and tolerability of the combination.

Hence, the present invention pertains also to a combination comprising a compound of formula 1, and at least one compound selected from the group consisting of lithium, divalproex, conventional antipsychotics, atypical anbpsychotics, lamotrigine and anti-depressants, in which the active ingredients are present in each case in free form or in the form of a pharmaceutically acceptable salt and optionally at least one pharmaceutically acceptable carrier, for simultaneous, separate or sequential use, especially for use in a method of treating affective disorders.

In one preferred embodiment of the present invention, a compound of formula I, wherein R₁ is hydrogen is combined with olanzapine. In another preferred embodiment of the present invention, a compound of formula I, wherein R₁ is hydrogen is combined with lithium or divalproex sodium.

Such a combination can be a combined preparation or a pharmaceutical composition.

The term “a combined preparation”, as used herein defines especially a “kit of parts” in the sense that the first and second active ingredient as defined above can be dosed independently or by use of different fixed combinations with distinguished amounts of the ingredients, i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the active ingredients. The ratio of the total amounts of the active ingredient 1 to the active ingredient 2 to be administered in the combined preparation can be varied, e.g., in order to cope with the needs of a patient sub-population to be treated or the needs of the single patient which different needs can be due to age, sex, body weight, etc. of the patients. Preferably, there is at least one beneficial effect, e.g., a mutual enhancing of the effect of the first and second active ingredient, in particular a synergism, e.g. a more than additive effect, additional advantageous effects, less side effects such as hypothyroidism, renal impairment, tremor, excess thirst, polyuria, GI side effects, nausea, extra-pyramidal symptoms, weight gain, cognitive dulling, fatigue, somnolence, sexual dysfunction and, especially, sedation than the single combination partners when applied in therapeutic effective dosages, a combined therapeutic effect in a non-effective dosage of one or both of the first and second active ingredient, and especially a strong synergism the first and second active ingredient.

Hence, the present invention also provides
the use of a combination as disclosed herein for the preparation of a medicament for the treatment of affective disorders; and
a commercial package comprising a combination as disclosed herein together with instructions for simultaneous, separate or sequential use thereof, especially for the treatment of affective disorders.

A combination comprising a compound of formula I, and at least one compound selected from the group consisting of lithium, divalproex, conventional anti-psychotics and atypical anti-psychotics, in which the active ingredients are present in each case in free form or in the form of a pharmaceutically acceptable salt and optionally at least one pharmaceutically acceptable carrier, for simultaneous, separate or sequential use, is especially useful for the treatment of mania.

A combination comprising a compound of formula I, and at least one antidepressants, in which the active ingredients are present in each case in free form or in the form of a pharmaceutically acceptable salt and optionally at least one pharmaceutically acceptable carrier, for simultaneous, separate or sequential use, is especially useful for the treatment of bipolar disorders. The combinations described herein are also in particular suitable for antimanic treatments.

When the combination partners employed in the combinations as disclosed herein are applied in the form as marketed as single drugs, their dosage and mode of administration can take place in accordance with the information provided on the package insert of the respective marketed drug in order to result in the beneficial effect described herein, if not mentioned herein otherwise. In particular, the following dosages can be administered to the patient:

Haloperidol may be administered to a patient in a total daily dosage of between about 5 to 25 mg.

Lithium can be administered to a patient in a total daily dosage of between about 0.5 to about 1 g.

Olanzapine can be administered to a patient in a total daily dosage of between about 2.5 to about 20 mg.

Quetiapine can be administered to a patient in a total daily dosage of between about 500 to about 600 mg.

Risperidone may be administered to a patient in a total daily dosage of between about 1 to about 6 mg.

Valproic acid sodium salt may be administered to a patient in a total daily dosage of between about 2000 to about 3000 mg.

In a preferred embodiment of the invention, the combination partners are applied in total dosages below the maximum dosages provided above, preferably in a total dosage representing less than 95% of the maximum dosages provided above, more preferably in a total dosage representing less than 75% of the maximum dosages provided above, even more preferably in a total dosage representing less than 50% of the maximum dosages provided above.

The following Examples serve to illustrate the invention without limiting the invention in its scope.

Abbreviations

Ac acetyl
aqu. aqueous
dansyl 5-(dimethylamino)-1-naphthalenesulfonyl
Et ethyl
HPLC high pressure liquid chromatography
Me methyl
NMR nuclear magnetic resonance
RT room temperature
THF tetrahydrofuran
Ts tosyl EXAMPLES

### Example 1

### Procedure for the Enantioselective Transfer Hydrogenation of 10-Oxo-10,11-dihydro-dibenzo[b,f]azepine-5-carboxylic acid amide to R(−)-10,11-Dihydro-10-hydroxy-5H-dibenz[b,f]azepine-5-carboxamide

To a mixture of 10-oxo-10,11-dihydro-dibenzo[b,f]azepine-5-carboxylic acid amide (300 mg, 1.189 mmol) and RuCl[(1R,2R)-p-TsNCH(C₆H₅)CH(C₆H₅)NH₂](η⁶-p-cymene, Aldrich, Switzerland) (8.8 mg, 0.0138 mmol) in CH₂Cl₂ (15 ml) is added dropwise a premixed solution of formic acid and NEt₃ (5:2, 328 mg:289 mg) at 23° C. and stirred for 10 min. The clear solution is heated to reflux for 16 h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford of R(−)-10,11-dihydro-10-hydroxy-5H-dibenzo[b,f]azepine-5-carboxamide (enantiomeric purity (ee) >99% determined by HPLC on Chiracel OD, Retention time: 9.46 min. [α]_{D}^{rt}=−195.3° (ethanol). ¹H-NMR (400 MHz, CDCl₃):7.70-7.20 (m, 8 H), 5.30 (br s,1 H), 5.10-4.60 (br s, 2 H), 3.75-3.40 (m, 1 H), 3.20-2.90 (m, 1 H), 2.50 (br s, 2 H). NMR-Datas refer to Lit.: Benes, J et al., J. Med. Chem. 1999, 42, 2582-2587. Molecular weight: 254.291

### Example 2

### Procedure for the Enantioselective Transfer Hydrogenation of 10-Oxo-10,11-dihydro-dibenzo[b,f]azepine-5-carboxylic acid amide to S(+)10,11-Dihydro10-hydroxy-5H-dibenz[b,f]azepine-5-carboxamide

To a mixture of 10-oxo-10,11-dihydro-dibenzo[b,f]azepine-5-carboxylic acid amide (300 mg, 1.189 mmol) and RuCl[(1S,2S)-p-TsNCH(C₆H₅)CH(CGH₅)NH₂](η⁶-p-cymene) (11 mg, 0.0173 mmol) in CH₂Cl₂ (15 ml) is added in two portions a premixed solution of formic acid and NEt₃ (5:2, 656 mg:578 mg) at 23° C. and stirred for 10 min. After that formic acid is added (50 μl) and the clear solution is heated to reflux for 16 h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford of S(+)-10,11-dihydro-10-hydroxy-5H-dibenzo[b,f]azepine-5-carboxamide (ee >99% by HPLC on Chiracel OD). Retention time: 12.00 min. [a]_{D}^{rt}=+196.6° (ethanol). ¹H-NMR (400 MHz, CDCl₃):7.70-7.20 (m, 8 H), 5.30 (br s,1 H), 5.10-4.60 (br s, 2 H), 3.75-3.40 (m, 1 H), 3.20-2.90 (m, 1 H), 2.50 (br s, 2 H). NMR-Datas refer to Lit.: Benes, J et al., J. Med. Chem. 1999, 42, 2582-2587. Molecular weight: 254.291

Alternative production: To a mixture of 10-oxo-10,11-dihydro-dibenzo[b,f]azepine-5-carboxylic acid amide (300 mg, 1.189 mmol) and RuCl[(1S,2S)-p-dansyl-NCH(C₆H₅)CH(C₆H₅)NH₂](η⁶-p-cymene) (8.5 mg, 0.012 mmol) in CH₂Cl₂ (15 ml) is added dropwise a premixed solution of formic acid and NEt₃ (5:2, 328 mg:289 mg) at 23° C. and stirred for 10 min. The clear solution is heated to reflux for 16 h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford of S(+)-10,11-dihydro-10-hydroxy-5H-dibenzo[b,f]azepine-5-carboxamide.

### Example 3

### Preparation of RuCl[(1S,2S)-p-dansylNCH(C₆H₅)CH(C₆H₅)NH₂](η⁶-p-cymene)

a) Preparation of (S,S)-5-dimethylaminonaphthalene-1-sulfonic acid (2-amino-1,2-diphenyl-ethyl)-amide: To a solution of (S,S)-diphenylethylenediamine (250 mg, 1.2 mmol) and triethylamine (0.5 ml) in THF is added dropwise a solution of dansyl chloride (318 mg, 1.2 mmol) in THF (2 ml) at 0° C. After stirring 16 h at RT the solvent is removed in vacuum and the residue is resolved in methylenchloride (20 ml). The organic solution is washed with NaHCO₃ solution (5 ml), dried over Na₂SO₄ and after filtration the solvent is removed. Flash chromatographie afford (S,S)-5-dimethylamino-naphthalene-1-sulfonic acid (2-amino-1,2-diphenyl-ethyl)-amide as yellow oil which crystallizes by drying in vacuum. M: 445.59. ¹H-NMR (400 MHz, CDCl₃):8.36 (t, J=7.5 Hz, 2 H), 8.17 (dd, J=7.2, 1.2 Hz, 1 H), 7.47 (dd, J=8.8 Hz, 1 H), 7.34 (dd, J=8.5 Hz, 1 H), 7.24-7.16 (m, 4 H), 7.11 (d, J=7.5 Hz, 1 H), 6.99-6.74 (m, 6 H), 4.61 (d, J=8.5 Hz, 1 H), 4.20 (d, J=8.5 Hz, 1 H), 2.80 (s, 6 H).

b) Preparation of RuCl[(1S,2S)-p-dansylNCH(C₆H₅)CH(C₆H₅)NH₂](η⁶-p-cymene): A solution of (S,S)-5-dimethylamino-naphthalene-1-sulfonic acid (2-amino-1,2-diphenyl-ethyl)-amide (80 mg, 0.18 mmol), NEt₃ (36 mg, 0.36 mmol) and [RuCl₂(p-cymene)]₂ (55 mg, 0.09 mmol) in 2-propanol is heated at 80° C. for 1 h. The solvent is removed after that und the dark red residue is washed with water (2 ml). The solid is dried in vacuum and used without any purification. M: 715.34.

### Example 4

### Vogel Conflict Test

Description of method: The method, which detects anxiolytic and related other psychiatric activity, follows that described by Vogel et al, Psychopharmacologia 1971 ;21:1-7. Anxiolytics and antidepressants (e.g., Fontana et al., Psychopharmacology 1989;98(2):157-62) of various classes increase punished drinking.

Rats are deprived of water for 48 hours and are then placed individually into a transparent Plexiglas enclosure (15×32×34 cm) with a floor consisting of stainless steel bars (0.4 cm) spaced 1 cm apart. The back wall of the enclosure is made of opaque Plexiglass thereby concealing the observer from the experimental animal. In the centre of the opposite wall, 5 cm above the floor, a metal water spout protrudes into the cage and is connected to one pole of a shock generator (Apelex: Type 011346). The other pole of the shock generator is connected to the metal grid floor. The rat is left to explore until it found the water spout. Then, every time it drinks, it receives a slight electric shock (1.7 mA, 1 sec.) 2 seconds after it starts lapping. The number of shocks received (punished drinkings) is counted during a 3 minute period. 15 rats are studied per group. The test is performed blind. Compounds are evaluated at 50, 100 and 200 mg/kg, administered p.o. 60 minutes before the test, and compared with a vehicle control group. Clobazam (64 mg/kg), administered under the same experimental conditions, is used as reference substance. All substances are evaluated within the same experiment and compared with the same vehicle and reference substance controls. Data are analyzed by comparing treated groups with vehicle control using unpaired Student's t tests.

1. A method for the treatment of affective disorders in a subject in need of such treatment, which comprises administering to said subject a therapeutically effective amount of a compound of formula I 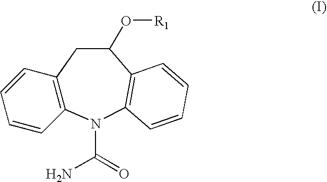
wherein R₁ represents hydrogen or C₁-C₃alkyl carbonyl.

2. The method according to claim 1, which comprises administering to said subject every 20 to 28 hours an amount between about 500 and about 3000 mg of a compound of formula 1, wherein R₁ represents hydrogen or C₁-C₃alkyl carbonyl.

3. The method according to claim 2, wherein every 20 to 28 hours an amount between 750 and 2500 mg of a compound of formula I is administered.

4. The method according to claim 1, wherein the disorder is selected from severe acute mania and manic episodes of bipolar I disorder.

5. The method according to claim 4, wherein every 20 to 28 hours an amount between 1500 and 2500 mg of a compound of formula I is administered.

6. The method according to claim 1, wherein the subject is a patient with a history of rapid cycling, with psychotic features, euphoric mania or dysphoric mania.

7. A method for the treatment of manic symptoms in a subject in need of such treatment, which comprises administering to said subject a therapeutically effective amount of a compound of formula I 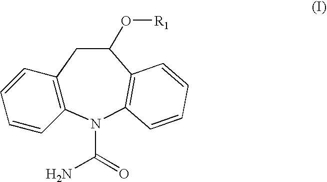
wherein R₁ represents hydrogen or C₁-C₃alkyl carbonyl.

8. A method for the maintenance treatment of affective disorders in a subject in need of such treatment, which comprises administering to said subject every a therapeutically effective amount of a compound of formula I 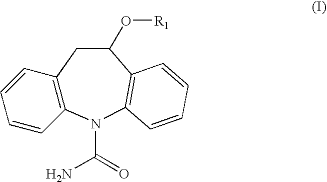
wherein R₁ represents hydrogen or C₁-C₃alkyl carbonyl.

9. The method according to claim 8, which comprises administering every 20 to 28 hours to said subject an amount between about 600 and about 2500 mg of a compound of formula I, wherein R₁ represents hydrogen or C₁-C₃alkyl carbonyl.

10. The method according to claim 9, wherein every 20 to 28 hours an amount between 750 and 1250 mg of a compound of formula I is administered.

11. The method according to claim 2, wherein R₁ represents hydrogen.

12. The method according to claim 1, wherein R₁ represents acetyl.

13-15. (canceled)

16. A pharmaceutical composition which incorporates as active agent a compound of formula I 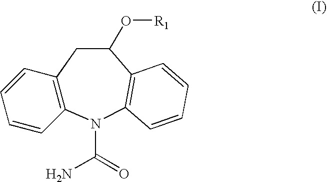
wherein R₁ represents hydrogen or C₁-C₃alkyl carbonyl or a pharmaceutically acceptable salt thereof, for use in the treatment of affective disorders.

17. A combination comprising
(a) a compound of formula I 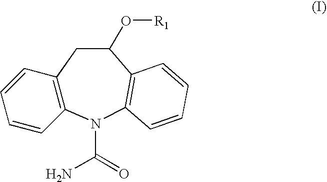
wherein R₁ represents hydrogen or C₁-C₃alkyl carbonyl, and
(b) at least one compound selected from the group consisting of lithium, divalproex, conventional antipsychotics, atypical antipsychotics, lamotrigine and antidepressants,
in which the active ingredients are present in each case in free form or in the form of a pharmaceutically acceptable salt,
and optionally at least one pharmaceutically acceptable carrier.

18. The combination according to claim 17, comprising a compound of formula I, wherein R₁ is hydrogen, and olanzapine.

19. The combination according to claim 17, comprising a compound of formula I, wherein R₁ is hydrogen, and a compound selected from lithium or divalproex sodium.

20. (canceled)

21. A commercial package comprising a combination according to claims 17 together with instructions for simultaneous, separate or sequential use thereof in the treatment of affective disorders.
